(19) United States
(12) Patent Application Publication
(10) Pub. No.: US 2012/0059337 A1
(43) Pub. Date: Mar. 8, 2012

(54) CATHETER WITH ASYMMETRIC OR COLLAPSIBLE-EXPANDABLE CROSS-SECTION

(76) Inventor: Eran Eilat, Herzliya (IL)

(21) Appl. No.: 13/221,994

(22) Filed: Aug. 31, 2011

Related U.S. Application Data
(60) Provisional application No. 61/378,946, filed on Sep. 1, 2010.
(60) Provisional application No. 61/392,978, filed on Oct. 14, 2010.

(51) Int. Cl. A61M 25/00

(52) U.S. Cl. 604/271; 604/523

(57) ABSTRACT

A catheter including a continuous envelope forming an open-bore shaft. The envelope is characterized by a main longitudinal axis and a sagittal cross section. At least a portion of the sagittal cross section may be characterized by at least one asymmetric property that extends along the main longitudinal axis in a predetermined pattern.

### FIELD OF THE INVENTION

The present invention generally pertains to a catheter with an asymmetric cross section and/or a shaft of collapsed and expanded cross sections.

### BACKGROUND OF THE INVENTION

Catheters are often used in the performance of medical procedures such as coronary angiography for injecting dye, or the like, into the cardiovascular system for diagnosis; and angioplasty to widen the lumen of a coronary artery which has become at least partially blocked by a stenotic lesion causing an abnormal narrowing of the artery due to injury or disease. In these techniques the distal end of the catheter is introduced into the aorta by way of the femoral artery. The proximal end of the catheter is then manipulated so its distal end is inserted into the lumen of a selected coronary artery branching off from the aorta. A typical angioplasty procedure would involve initially inserting a guiding catheter into the cardiovascular system in the above manner, followed by a dilating catheter, a laser catheter, an atherectomy catheter, or the like, which is guided through the guiding catheter until its distal end portion is positioned within the stenotic lesion in the coronary artery to reduce the blockage in the artery. A diagnostic catheter would be used in the same manner. More details are elaborated in U.S. Pat. Nos. 5,445,625, 6,083,213 and WO0232495A1 which are incorporated herein as a reference. For guiding catheters see more in the following citations, that are incorporated herein as a references: Meier B. Percutaneous coronary intervention. In: Topol E J (ed), Textbook of Cardiovascular Medicine, 2nd edition. 2002, pp. 1665-1676; Kiemeneij F, Laarman G J, de Melker E. Transradial artery coronary angioplasty. Am Heart J 1995; 129:1-7; and Campeau L. Percutaneous radial artery approach for coronary angiography. Cathet Cardiovasc Diagn 1989; 16:3-7.

The insertion of guide catheters and the like into body cavities, including non-uniform body cavities, such as branched arteries during coronary angioplasty and similar procedures is involved with considerable risk, evan a risk of death, as disclosed in WO2008005388A2, U.S. Pat. No. 6,960,222, U.S. Pat. No. 6,936,060, U.S. Pat. No. 6,908,474, U.S. Pat. No. 6,905,490, U.S. Pat. No. 6,682,505, U.S. Pat. No. 6,645,222, U.S. Pat. No. 6,632,236, U.S. Pat. No. 6,582,396, U.S. Pat. No. 6,540,712 U.S. Pat. No. 6,423,032, U.S. Pat. No. 6,413,235, and U.S. Pat. No. 6,206,868 which are all incorporated herein as a reference.

Current catheters, such as urethral catheters, Foley catheters etc. are characterized by a large diameter (e.g., about 9 mm) relative to the urethral lumen. This is a cause of discomfort, fear, pain and trauma for the patient, while the insertion of such a wide and flexible catheter is a difficult procedure for less experienced physicians.

There is a long felt and unmet need for an improved catheter which is h easy to insert and still has a wide enough bore. Moreover, easy methods and apparatus for insertion of catheters and similar inserts from within a body cavities, namely non-uniform body cavity, such as branched arteries, that simultaneously reduce the risk of injuring the body tissue are still a long felt and unmet need.

### BRIEF DESCRIPTION OF THE FIGURES

The objects and advantages of various embodiments of the invention will become apparent from the following description when read in conjunction with the accompanying drawings wherein:

FIG. 1A and FIG. 1B schematically illustrate in an-out-of scale manner a distal portion of a commercially available catheter known in the art and a cross section A:A of the aforesaid commercially available catheter;

FIG. 2a to FIG. 2h and FIG. 3, schematically illustrate in an-out-of scale manner transverse cross sections (FIG. 2a to FIG. 2f) and a sagittal cross section (FIG. 3) of various embodiments of the novel catheter as defined in the present invention;

FIG. 4, schematically illustrate in an-out-of scale manner a side view of a distal portion of a catheter;

FIG. 5, schematically illustrate in an-out-of scale manner a cross section of a commercially available catheter;

FIG. 6a to FIG. 6d, schematically illustrate in an-out-of scale manner a transverse cross sections (FIG. 6a to FIG. 6c) and a sagittal cross section (FIG. 6d) of the novel catheter as defined in the present invention; and

FIG. 7a and FIG. 7b, schematically illustrate in an-out-of scale manner an embodiment of the novel catheter as defined in the present invention.

### SUMMARY OF THE INVENTION

It is one object of the present invention to provide a catheter having at least one characteristic being asymmetric with respect to least one plane of said catheter, wherein said characteristic provides said catheter with maneuverability within a body cavity.

It is another object of the present invention to provide catheter comprising a continuous envelope forming an open-bore shaft; said envelope is characterized by a main longitudinal axis and a sagittal cross section; wherein at least a portion of said sagittal cross section is characterized by at least one asymmetric property; further wherein said asymmetric property extends along said main longitudinal axis according to predetermined pattern.

It is another object of the present invention to provide a catheter comprising a continuous envelope forming an open-bore shaft; said envelope is characterized by a main longitudinal axis and a corneal cross section; wherein at least a portion of said corneal cross section is characterized by at least one asymmetric property; further wherein said asymmetric property extends along said main longitudinal axis according to predetermined pattern.

It is another object of the present invention to provide a catheter comprising a continuous envelope forming an open-bore shaft; said envelope is characterized by a main longitudinal axis and a transverse cross section; wherein at least a portion of said transverse cross section is characterized by at least one asymmetric property; further wherein said asymmetric property extends along said main longitudinal axis according to predetermined pattern.

It is another object of the present invention to provide a catheter comprising a continuous envelope forming an open-bore shaft; said envelope is characterized by a main longitudinal axis and a (i) sagittal cross section; (ii) corneal cross section; (iii) transverse cross section or any combination thereof; wherein at least a portion of either (i) said sagittal cross section; (ii) corneal cross section; (iii) transverse cross section or any combination thereof, is characterized by at least one asymmetric property; further wherein said asymmetric property extends along said main longitudinal axis according to predetermined pattern. It is another object of the present invention to provide the catheter as defined above, wherein said predetermined pattern is selected from a group consisting of parallel manner or a non-parallel manner.

It is another object of the present invention to provide the catheter as defined above, wherein said predetermined pattern is a coiled or helix like pattern in either clock wise or counter clock wise manner.

It is another object of the present invention to provide the catheter as defined above, wherein said catheter is insertable into a non-uniform body cavity.

It is another object of the present invention to provide the catheter as defined above, wherein said portion of said cross section comprises at least one bulge protruding either inwardly or outwardly of said envelope or any combination thereof.

It is another object of the present invention to provide the catheter as defined above, wherein said protruding bulge protrudes to a range of about 0.01 millimeters to about 2.5 millimeters.

It is another object of the present invention to provide the catheter as defined above, wherein the thickness of said at least one bulge is substantially different than the thickness of said envelope.

It is another object of the present invention to provide the catheter as defined above, wherein said bulge is located on the perimeter of said envelope.

It is another object of the present invention to provide the catheter as defined above, wherein said portion of said envelope is made of material having predetermined Property Y1 while said envelope is made of material having predetermined Property Y2; where Y1 is substantially different than Y2.

It is another object of the present invention to provide the catheter as defined above, wherein said Property is selected from a group consisting of Young's modulus, rigidity, flexibility, shear modulus G, bulk modulus K, Poisson's ratio v, spring modulus, bending stiffness El, thickness, texture, composition, electrical conductivity, density, composition, elasticity.

It is another object of the present invention to provide the catheter as defined above, wherein said Young's modulus is in the range of about 0.01 to about 0.05 GPa.

It is another object of the present invention to provide the catheter as defined above, wherein said material is selected from a group consisting of Latex™, silicon containing material, shape memory materials, electro active materials or any combination thereof.

It is another object of the present invention to provide the catheter as defined above, wherein said envelope is made of at least one material selected from a group consisting of Latex™, silicon containing material, shape memory materials, electro active materials or any combination thereof.

It is another object of the present invention to provide the catheter as defined above, wherein said portion of said cross section comprises at least one balloon-style bulge, such that said asymmetric property is provided by wither inflating of deflating said balloon.

It is another object of the present invention to provide the catheter as defined above, wherein said asymmetric property is either automatically controlled or manually controlled by the user.

It is another object of the present invention to provide the catheter as defined above, wherein said portion of said cross section comprises either at least one balloon-style bulge; or at least one bulge or any combination thereof.

It is another object of the present invention to provide the catheter as defined above, wherein said asymmetric property is provided by means of predefined microstructure texture.

It is another object of the present invention to provide the catheter as defined above, wherein said at least one property is selected from a group consisting of size, texture, material from which said envelope is made of, mechanical properties, mechanical properties selected from a group consisting of rigidity, flexibility, Young's modulus E, shear modulus G, bulk modulus K, Poisson's ratio v, spring modulus, bending stiffness El, thickness, texture, composition, electrical conductivity, density, composition, elasticity or any combination thereof.

It is another object of the present invention to provide the catheter as defined above, wherein linear reciprocation and/or rotatable maneuver of the proximal end of said shaft translates into movement of said catheter in said body cavity.

It is another object of the present invention to provide a method of treating a patient, said method comprising step of:
a. obtaining a catheter having a continuous envelope which forms an elongated open-bore shaft; said envelope is characterized by a main longitudinal axis and a sagittal cross section;
b. asymmetrically modifying at least one property in at least a portion of said sagittal cross section;
c. exceeding said section along said shaft according to predetermined pattern; and,
d. inserting said catheter into a body cavity; and,
e. treating said patient.

It is another object of the present invention to provide a method of treating a patient, said method comprising step of:
a. obtaining a catheter having a continuous envelope which forms an elongated open-bore shaft; said envelope is characterized by a main longitudinal axis and a corneal cross section;
b. asymmetrically modifying at least one property in at least a portion of said corneal cross section; and,
c. exceeding said section along said shaft according to predetermined pattern;
d. inserting said catheter into a body cavity; and,
e. treating said patient.

It is another object of the present invention to provide a method of treating a patient, said method comprising step of:
a. obtaining a catheter having a continuous envelope which forms an elongated open-bore shaft; said envelope is characterized by a main longitudinal axis and a transverse cross section;
b. asymmetrically modifying at least one property in at least a portion of said transverse cross section;
c. exceeding said section along said shaft according to predetermined pattern; and,
d. inserting said catheter into a body cavity; and,
e. treating said patient.

It is another object of the present invention to provide a method of treating a patient, said method comprising step of:
a. obtaining a catheter having a continuous envelope which forms an elongated open-bore shaft; said envelope is characterized by a main longitudinal axis and a (i) transverse cross section; (ii) sagittal cross section; (iii) sagittal cross section;
b. asymmetrically modifying at least one property in at least a portion of either (i) (i) transverse cross section; (ii) said sagittal cross section; (iii) said sagittal cross section; or any combination thereof;
c. exceeding said section along said shaft according to predetermined pattern; and,
d. inserting said catheter into a body cavity; and,
e. treating said patient.

It is another object of the present invention to provide the methods as defined above, additionally comprising step of selecting said predetermined pattern from a group consisting of parallel manner or a non-parallel manner.

It is another object of the present invention to provide the methods as defined above, additionally comprising step of selecting predetermined pattern to be a coiled or helix like pattern in either clock wise or counter clock wise manner.

It is another object of the present invention to provide the methods as defined above, additionally comprising step of inserting said catheter into a non-uniform body cavity.

It is another object of the present invention to provide the methods as defined above, additionally comprising step of providing said catheter with at least one bulge in said portion of said cross section; said bulge is protruding either inwardly or outwardly of said envelope or any combination thereof.

It is another object of the present invention to provide the methods as defined above, wherein said protruding bulge protrudes to a range of about 0.01 millimeters to about 2.5 millimeters.

It is another object of the present invention to provide the methods as defined above, additionally comprising step of providing said catheter such that the thickness of said at least one bulge is substantially different than the thickness of said envelope.

It is another object of the present invention to provide the methods as defined above, additionally comprising step of providing said catheter wherein said bulge is located on the perimeter of said envelope.

It is another object of the present invention to provide the methods as defined above, additionally comprising step of providing said catheter such that said portion of said envelope is made of material having predetermined Property Y1 while said envelope is made of material having predetermined Property Y2; where Y1 is substantially different than Y2.

It is another object of the present invention to provide the methods as defined above, additionally comprising step of selecting said Property from a group consisting of Young's modulus, rigidity, flexibility, shear modulus G, bulk modulus K, Poisson's ratio v, spring modulus, bending stiffness El, thickness, texture, composition, electrical conductivity, density, composition, elasticity.

It is another object of the present invention to provide the methods as defined above, wherein said Young's modulus is in the range of about 0.01 to about 0.05 GPa.

It is another object of the present invention to provide the methods as defined above, additionally comprising step of selecting said material from a group consisting of Latex™, silicon containing material, shape memory materials, electro active materials or any combination thereof.

It is another object of the present invention to provide the methods as defined above, additionally comprising step of providing said envelope from of at least one material selected from a group consisting of Latex™, silicon containing material, shape memory materials, electro active materials or any combination thereof.

It is another object of the present invention to provide the methods as defined above, additionally comprising step of providing said portion of said cross section with at least one balloon-style bulge, such that said asymmetric property is provided by wither inflating of deflating said balloon.

It is another object of the present invention to provide the methods as defined above, wherein said asymmetric property is either automatically controlled or manually controlled by the user.

It is another object of the present invention to provide the methods as defined above, additionally comprising step of providing said portion of said cross section with either at least one balloon-style bulge; or at least one bulge or any combination thereof.

It is another object of the present invention to provide the methods as defined above, additionally comprising step of providing said asymmetric property by means of predefined microstructure texture.

It is another object of the present invention to provide the methods as defined above, additionally comprising step of selecting said at least one property from a group consisting of size, texture, material from which said envelope is made of, mechanical properties, mechanical properties selected from a group consisting of rigidity, flexibility, Young's modulus E, shear modulus G, bulk modulus K, Poisson's ratio v, spring modulus, bending stiffness El, thickness, texture, composition, electrical conductivity, density, composition, elasticity or any combination thereof.

It is another object of the present invention to provide the methods as defined above, additionally comprising step of activating the proximal end of said shaft such that within a branched and/or non-linear body cavity of a defined bore size and structure, junctions and braches thereof, said activation is advanced towards and within said junctions or braches

It is another object of the present invention to provide the methods as defined above, wherein said step of activating the proximal end of said shaft additionally comprising careful linear reciprocation and/or rotatable maneuver or any combination thereof.

It is still an object of the present invention to provide the methods as defined above, additionally comprising step of inserting said catheter into a body cavity,

It is another object of the present invention to provide the methods as defined above, wherein said body cavity is non-uniform body cavity.

Moreover, it is further in the scope of invention to describe a novel catheter, provided with reversibly collapsible & expandable configurations. The catheter has, inter alia, in its distal portion, a continuous envelope forming an elongated open-bore shaft insertable within a body cavity. At least one cross section of this shaft and/or tip, i.e., at least one portion along the shaft's length or along its entire length, comprises at least one reversibly invaginated sector. These one or more invaginated sectors provide the catheter with the required (i) at least one collapsible configuration, characterized by a relatively rigid shaft and small cross section, and (ii) at least one expandable configuration characterized by a flexible shaft and/or tip and larger cross section.

It is also in the scope of invention wherein, in the aforesaid collapsible configuration, at least one sector is reversibly held in its collapsed (invaginated) configuration by means of at least one insert. For example, the insert is adapted for mechanically clutching the invaginated envelope, tying it or otherwise reversibly affixing the envelope wall in the invaginated, encapsulated, shrunken, or contracted state.

It is further in the scope of invention to describe a novel method of treating a patient by means of the catheter as defined and described in the present invention. The method comprises steps of obtaining a catheter having a continuous envelope which forms an elongated open-bore shaft; providing at least one cross section of the shaft with one or more reversibly invaginated sectors; inserting the catheter, in its at least one collapsible configuration, within a body cavity whilst maintaining the shaft with a relative rigidity and small cross section; and then, when required, still within the cavity, expanding the cross section of at least one portion of the catheter to its expanded configuration whilst maintaining the flexibility of the shaft and with larger cross section.

It is further in the scope of invention to disclose the method as defined above, wherein this method additionally comprises steps selected from (i) maintaining the shaft and/or tip in its collapsible configuration by providing an insert to effectively clutch the invaginated envelope within the sector, and (ii), expanding the shaft to its expanded configuration by manipulating the insert to disengage from the invaginated envelope, such that a flexible shaft with large cross section is obtained.

It is further in the scope of invention to disclose a catheter having a continuous envelope forming an elongated open-bore shaft and/or tip insertable within a body cavity. The cross section of the shaft is reciprocally maneuverable form at least one collapsible configuration of diameter d to at least one expandable configuration of diameter D, wherein D>d. Diameters d and D may range from about 0.01 mm to about 25 mm and from about 0.05 mm to about 50, respectively. In a latex urinary catheter as defined in the present invention, d ranges e.g., from about 2 to about 6 mm; and D ranges e.g., from about 6 to about 13 mm. It is further in the scope of invention to disclose a catheter having a continuous envelope forming an elongated open-bore shaft and/or tip insertable within a body cavity. The cross section of the shaft is reciprocally maneuverable form at least one collapsible configuration of cross section area a to at least one expandable configuration of cross section area A, wherein A>a. In a latex urinary catheter as defined in the present invention, a is about e.g., 30 mm² and A is about e.g., 201 mm².

It is also in the scope of invention to disclose wherein the at least one portion of the shaft characterized by a collapsible cross section is reversibly held in its collapsed/expended configuration by means of at least one insert.

It is also in the scope of invention to disclose a method of treating a patient. The method comprises a step of obtaining a catheter having a continuous envelope which forms an elongated open-bore shaft and/or tip; providing at least one portion of the shaft and/or tip with a reciprocal maneuverability to change from at least one collapsed configuration to at least one expand configuration, by providing the at least one portion with at least one collapsible configuration, characterized by a cross section area a, and at least one expandable configuration characterized by cross section area A; A>a; inserting the catheter, in its at least one collapsible configuration, within a body cavity whilst maintaining the shaft with a relative rigidity and small cross section; and then, still within the cavity, expanding the cross section of at least one portion of the catheter to its expanded configuration whilst maintaining the shaft flexible and with larger cross section.

It is further in the scope of invention to disclose a method as defined above, wherein the method additionally comprises steps selected from (i) maintaining the shaft and/or tip in its collapsible configuration by providing an insert to effectively clutch the portion of the shaft, and (ii) expanding the shaft and/or tip to its expanded configuration by manipulating the insert to stop clutching, such that a flexible shaft with large cross section is obtained.

It is further in the scope of invention to disclose a method of treating a patient. The method comprises a step of obtaining a catheter having a continuous envelope which forms an elongated open-bore shaft and/or tip; providing at least one portion of the shaft and/or tip with a reciprocal maneuverability to change from at least one collapsed configuration to at least one expand configuration, by providing the at least one portion with at least one collapsible configuration, characterized by diameter d, and at least one expandable configuration characterized by diameter D; D>d; inserting the catheter, in its at least one collapsible configuration, within a body cavity whilst maintaining the shaft with a relative rigidity and small cross section; and then, still within the cavity, and expanding the cross section of at least one portion of the catheter to its expanded configuration whilst maintaining the shaft flexible and with larger cross section.

It is further in the scope of invention to disclose a method as defined above, wherein the method additionally comprises steps selected from (i) maintaining the shaft and/or tip in its collapsible configuration by providing an insert to effectively clutch the portion of the shaft, and (ii) expanding the shaft and/or tip to its expanded configuration by manipulating the insert to stop clutching, such that a flexible shaft with large cross section is obtained.

### DETAILED DESCRIPTION OF THE PREFERRED EMBODIMENTS

The following description is provided, alongside all chapters of the present invention, to enable any person skilled in the art to make use of the invention and sets forth the best modes contemplated by the inventor of carrying out this invention. Various modifications, however, will remain apparent to those skilled in the art, since the generic principles of the present invention have been defined specifically to provide a catheter with reversibly provided collapsible & expanded configurations as herein defined and described.

It is thus one object of the present invention to provide a catheter having at least one characteristic being asymmetric with respect to least one plane of said catheter, wherein said characteristic provides said catheter with maneuverability within a body cavity.

It is another object of the present invention to provide catheter comprising a continuous envelope forming an open-bore shaft; said envelope is characterized by a main longitudinal axis and a sagittal cross section; wherein at least a portion of said sagittal cross section is characterized by at least one asymmetric property; further wherein said asymmetric property extends along said main longitudinal axis according to predetermined pattern.

It is another object of the present invention to provide a catheter comprising a continuous envelope forming an open-bore shaft; said envelope is characterized by a main longitudinal axis and a corneal cross section; wherein at least a portion of said corneal cross section is characterized by at least one asymmetric property; further wherein said asymmetric property extends along said main longitudinal axis according to predetermined pattern.

It is another object of the present invention to provide a catheter comprising a continuous envelope forming an open-bore shaft; said envelope is characterized by a main longitudinal axis and a transverse cross section; wherein at least a portion of said transverse cross section is characterized by at least one asymmetric property; further wherein said asymmetric property extends along said main longitudinal axis according to predetermined pattern.

It is another object of the present invention to provide a catheter comprising a continuous envelope forming an open-bore shaft; said envelope is characterized by a main longitudinal axis and a (i) sagittal cross section; (ii) corneal cross section; (iii) transverse cross section or any combination thereof; wherein at least a portion of either (i) said sagittal cross section; (ii) corneal cross section; (iii) transverse cross section or any combination thereof, is characterized by at least one asymmetric property; further wherein said asymmetric property extends along said main longitudinal axis according to predetermined pattern.

The term ‘catheter’ refers herein to any medically or veterinary used catheter, and to any tube or the like that can be inserted into a body cavity, duct, or vessel and allow drainage, injection of fluids, or access by surgical instruments. The catheter is at least partially made of flexible matter such as, but not limited to: latex, silicone-containing materials, rubber, polymers, Gor-tex™ ore any other polytetrafluoroethylene (PTFE), or the like and any combination and mixtures thereof. The catheter is possibly made or contain radiopaque dyes or agents thereof.

It is in the scope of the invention wherein the term ‘catheter’ or ‘guiding catheter’ as used herein below interchangeably refers to catheters, tubes, guide wires, trocars, intravenal pipings, inserts, needles, endoscopes and sheaths thereof utilized in human or veterinary surgery, such as percutaneous coronary intervention (PCI), percutaneous transluminal coronary angioplasty (PTCA), coronary artery bypass surgery (CABG), balloon angioplasty, urological procedures etc.

The term ‘body cavity’ refers interchangeably herein to both natural body cavities and orifices thereof, such as the circulatory system, blood vessels such as arteries, branches of the aorta, such as the carotid artery, the subclavian artery, the celiac trunk, the mesenteric arteries, etc the renal artery and the iliac artery, arties, coronary circulation, blood vessels of the coronary circulation, urethra, vagina, anus, airways, or other NOTS openings, and to unnatural body openings, such as facilitatebly provided openings, such as in laparoscopic surgery procedures, intravascular canula etc.

The term ‘non-uniform body cavity’ refers a body cavity as defined above, having at least one irregular shape, such as branches, junctions, cavities; or alternatively, one or more narrowing or constricting sections, strictions; or alternatively, broadening or widening sections, enlargements, expansions, dilations, evolvements, expansionisms, expansiveness, extensiveness, aggrandizements, commodiousness, dilatations etc; or alternatively curved, fickle or crooked sections.

The term ‘latex’ refers herein to either natural or synthetic stable dispersions (emulsion) of polymer microparticles in an aqueous medium, such as rubber latex. In a wider spectrum, the term is referring any flexible biocompatible material, such as rubber, polyamide (Nylon™), polyalkene or the like, silicone etc.

The term ‘asymmetric cross section’ refers herein to an asymmetry provided in the catheter, inter alia, in one or more of its following cross-sections, namely: sagittal, corneal or transverse plans. The term also refers to any asymmetry provided in other planes of the shaft. The asymmetry of the catheter is provided by various means; such as by constructing at least one section of the catheter with a material characterized by at least one character, and this at least one character is different form at least one character of other portions of the catheter. Hence for example, the at least one character is selected from a group consisting of rigidity, flexibility, Young's modulus E, shear modulus G, bulk modulus K, Poisson's ratio v, spring modulus, bending stiffness El, thickness, texture, composition, electrical conductivity, density, method of manufacture and any combination of the same.

The term ‘about’ refers herein a value being±25% of the defined measure.

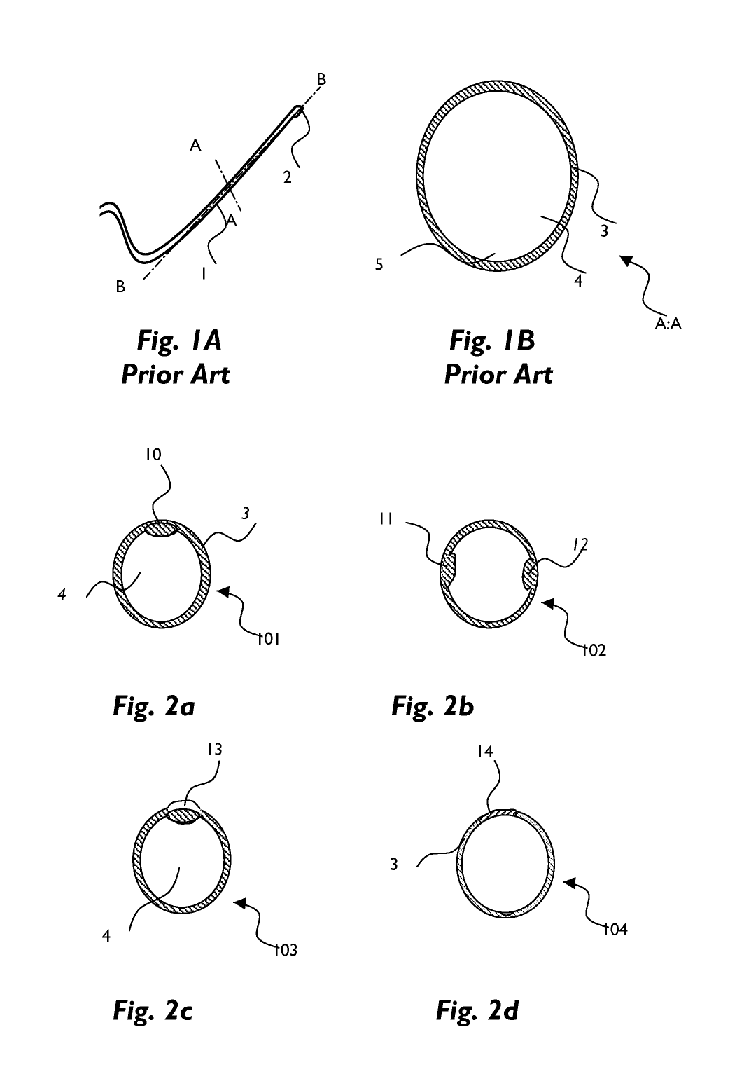

Reference is now made to FIG. 1a, presenting a side and not-in-scale illustration of a distal portion of a commercially available catheter known in the art, having an insertion tips (2) in its very distal end and an elongated shaft (neck) portion (1). Transverse cross section A:A defines at least one section along the shaft or tip. The main elongated axis of the shaft is marked in dashed line B:B.

Reference is now made to FIG. 1b, illustrating the cross section A:A of the aforesaid commercially available catheter. This view, provided here in a not-in-scale manner, schematically presents one portion of the aforesaid elongated shaft. The cross section shows a relatively larger open bore 4. The thickness of the envelope (walls, 3) of the catheter is substantially smooth, flexible, uni-layered, homogeneous and thin catheter.

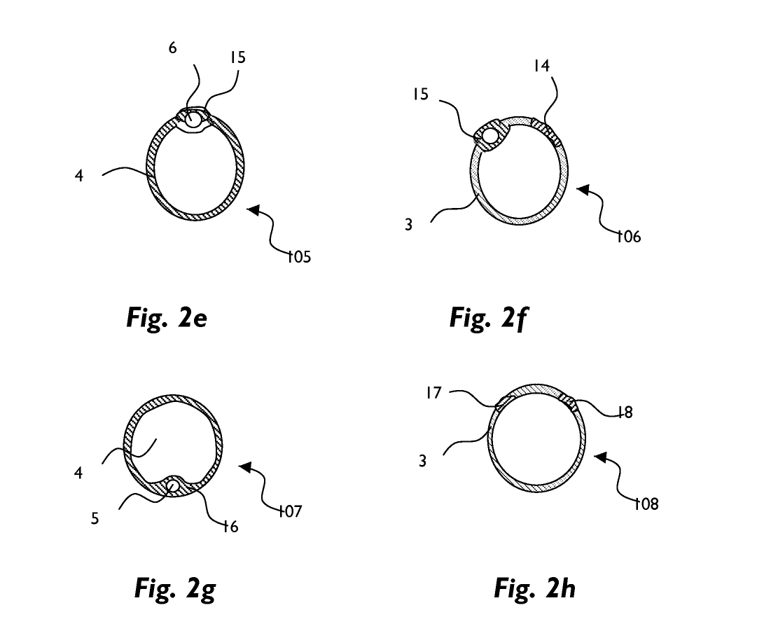

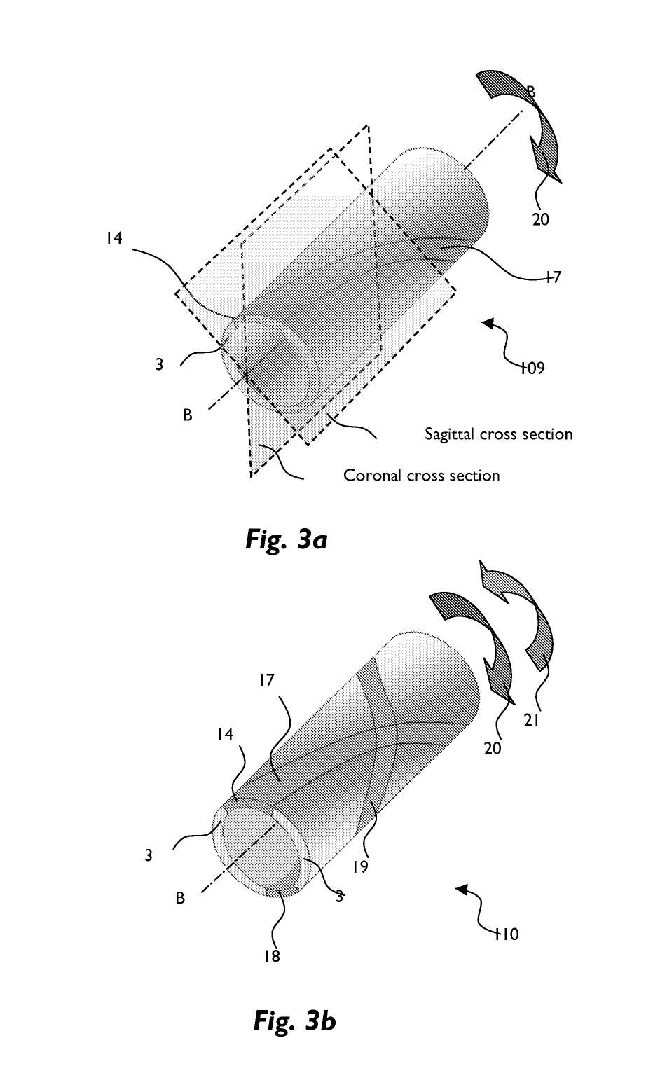

Reference is now made to FIG. 2a to FIG. 2h and FIG. 3, schematically illustrating, still in a non-in-scale manner, transverse cross sections (FIG. 2a to FIG. 2f) and a sagittal cross section (FIG. 3) various embodiments of the novel catheter as defined in the present invention.

FIG. 2a thus illustrates a cross section of one possible embodiment (101) of the catheter according to the present invention. The shaft comprises a relatively larger open bore 4, suitable for fluid delivery. Like prior art, the thickness of most of the envelope (walls, 3) of the catheter is substantially smooth, uni-layered, homogeneous and thin, so as to provide the catheter with flexibility. The catheter yet further comprising a bulge 10, here a section of a relatively thick wall protruding inwardly, i.e., into bore 4. Said at least one less-flexible section 10 is located anywhere along the perimeter of the envelope. Section 10 is less flexible than envelope 3, and thus is provides a structural element. A coiled ridge (See e.g., portion 17 in FIG. 3) of bulge 10 with encircles the main elongated axis B:B of the shaft, provides a rotation of the tip portion 2 and/or the shaft portion 1 of the catheter when the catheter is linearly maneuvered within the body cavity.

The catheter of the present invention is characterized by a tip portion 2 and/or at last one shaft portion 1 having either a continuous or controlled asymmetry or tendency (flexibility) to escape from the preset perimeter of the envelope. As an example, and in a non-limiting manner, the bulge is protruding the circumference of the envelope in a measure ranging from about 0.01 and less millimeters to about 2.5 and more millimeters. The asymmetry is provided, inter alia and according to yet another embodiment, by means of a predefined micro-structured texture.

Hence, within a branched and/or non-linear body cavity of a defined bore size and structure, junctions and braches thereof, a careful linear reciprocation and/or rotatable maneuver of the shaft 1 in its proximal end, tip portion 2 and/or at last one shaft portion 1 is advanced towards and within said junctions or braches.

Reference is now made to FIG. 2b which illustrates a cross section of another possible embodiment (102) of the catheter of the present invention. The catheter comprising here more than one bulge, here two bulges 11 and 12 of relatively thick walls which protrude inwards.

Reference is now made to FIG. 2c which illustrates a cross section of another possible embodiment (103) of the catheter of the present invention. The catheter comprising here one bulge 13 of relatively thick walls which protrude inwards and outwards. It is well in the scope of the invention wherein more than one type bulges (13) are used, and wherein the bulge protrudes only outside the envelope perimeter.

Reference is now made to FIG. 2d which illustrates a cross section of another possible embodiment (104) of the catheter of the present invention. The catheter comprising here at least one section 14 of relatively either flexible or rigid wall section. This section is not protruding inwards nor outwards. For example, the envelope 3 is made of one material, e.g., at least one first Latex™ of predefined Young modulus, and portion 14 is made of at least one second Latex™ or silicone-containing materials having a different Young modulus. Hence for example and in a non-limiting manner, the at least one first Latex™ is characterized by a Young's modulus ranging e.g., from about 1,500 to about 7,000 and modulus ranging e.g., about 0.01 to about 0.05 GPa, and the at least one second Latex™ or silicone containing material is characterized by a Young's modulus ranging e.g., from about 7,500 to about 15,000 and modulus ranging e.g., about 0.05 to about 0.01 GPa, and vice versa.

Reference is now made to FIG. 2e which illustrates a cross section of another possible embodiment (105) of the hereby patented catheter. The catheter comprising at least one inflatable/deflateable balloon-style bulge 15 having at least one open bore 6. Here, the asymmetric proportions of the cross section are controlled by the operator. Inflation of the balloon increases asymmetry. It is in the scope of the invention wherein more than one controllable type bulges 15 are used, and wherein the bulge protrudes only outside the envelope perimeter.

It should be emphasized that due to the above mentioned asymmetry property, the catheter tip has a tendency to one side, which favors one of the directions (right, left, up, down).

Each time the operator desires to reverse the catheter tendency he inflates/deflates the balloon, and thus the catheter tip shall direct the opposite side.

Reference is now made to FIG. 2f which illustrates a cross section of another possible embodiment (106) of the hereby patented catheter. The catheter is characterized by heterogeneous asymmetric properties. Catheter 106 comprises, inter alia, (a) at least one inflatable/deflateable balloon-style bulge 15 having at least one inflatable volume 6; and (b) at least one section 14 of relatively either flexible or rigid wall section as compared with wall 3. Here again, the asymmetric proportions of the cross section are controlled by the operator.

Reference is now made to FIG. 2g which illustrates a cross section of another possible embodiment (107) of the hereby patented catheter. The shaft comprises a relatively larger open bore 4, suitable for fluid delivery, and a smaller bore 5, especially adapted from inflating and deflating a balloon located at the tip of the catheter, such as in urinary catheters. Here in catheter 107, the continuous envelope 16 surrounding bore 5 is designed to provide the required asymmetric characteristics. Referring again to FIG. 3, bore 5 provided by envelope 16 is not parallel to main longitudinal axis B:B yet it is coiled along the axis in a spiral wound manner defined, e.g., by line 17.

Reference is now made to FIG. 2g which illustrates a cross section of another possible embodiment (108) of the hereby disclosed catheter. The shaft is characterized by asymmetric proportions provided by using shape memory materials, especially Nitinol™ and electro-active polymers, located along the perimeter of the shaft, especially along its main longitudinal axis of one or more portions of the shaft, see e.g., a fish-skeleton-like design with sections 17 and 18 which comprises one or more Nitinol™-made rib-like structures.

Reference is now made to FIG. 3a which illustrates a three-dimension out-of-scale simplified presentation of one section of catheter 109 having an asymmetric configuration, the asymmetry is notable in the sagittal and coronial planes and cross-sections thereof. Referring again to shaft 104, wall 3 is made of one flexible material, e.g., at least one first Latex™ or one or more silicone-containing materials, of a defined Young modulus, wherein section 14 is made from another material or from a mixtures of materials. Section 14 exceed along the shaft in a coiled manner (see line 17), such that it coiled clock-wise over the main longitudinal axis B:B.

According to one embodiment of the invention, section 14 is made from at least one second Latex™ or silicone-containing materials having, as compared with wall 3, a different Young modulus. Hence for example and in a non-limiting manner, the at least one first Latex™ is characterized by a Young's modulus ranging e.g., from about 1,500 to about 7,000 and modulus ranging e.g., about 0.01 to about 0.05 GPa, and the at least one second Latex™ or silicone containing material is characterized by a Young's modulus ranging e.g., from about 7,500 to about 15,000 and modulus ranging e.g., about 0.05 to about 0.01 GPa, and vice versa. According to yet another embodiment of the invention, see description above, section 14 comprises one or more shape memory alloys or electro-active polymers. Hence, by applying an electrical current upon section 14, a rotational movement 20 is provided, here, a clock-wise (20) rotation over the main longitudinal axis B:B.

Reference is lastly made to FIG. 3b which illustrates a three-dimension out-of-scale simplified presentation of one section of catheter 110 having a complicated asymmetric configuration. Wall 3 is made e.g., of one flexible material, e.g., at least one first Latex™ or one or more silicone-containing materials, of a defined Young modulus, wherein section 14 is made e.g., of yet another material or a mixtures of materials. Section 14 exceed along the shaft in a coiled manner (see line 17), such that it coiled clock-wise over the main longitudinal axis B:B; and further wherein section 17 is made e.g., of yet another material or a mixtures of materials. Section 17 exceed along the shaft in a coiled manner (see line 19), such that it coiled counter clock-wise (21) over the main longitudinal axis B:B.

According to one embodiment of the invention, section 19 is made by at least one second Latex™ or silicone-containing materials having, as compared with wall 3, a different Young modulus. Hence for example and in a non-limiting manner, the at least one first Latex™ is characterized by a Young's modulus ranging e.g., from about 1,500 to about 7,000 and modulus ranging e.g., about 0.01 to about 0.05 GPa, and the at least one second Latex™ or silicone containing material is characterized by a Young's modulus ranging e.g., from about 7,500 to about 15,000 and modulus ranging e.g., about 0.05 to about 0.01 GPa, and vice versa. According to yet another embodiment of the invention, see description above, section 19 comprises one or more shape memory alloys or electro-active polymers. Hence, by applying an electrical current upon section 19, a rotational movement 21 is provided, here, a counter clock-wise rotation over the main longitudinal axis B:B. Shaft 110 is thus maneuverable to (i) counter clock-wise and/or (ii) clock-wise rotations.

Reference is now made to FIG. 4, presenting a side and not-in-scale illustration of a distal portion of a catheter, having an insertion tip (2) at its very distal end and an elongated shaft (neck) portion (1).

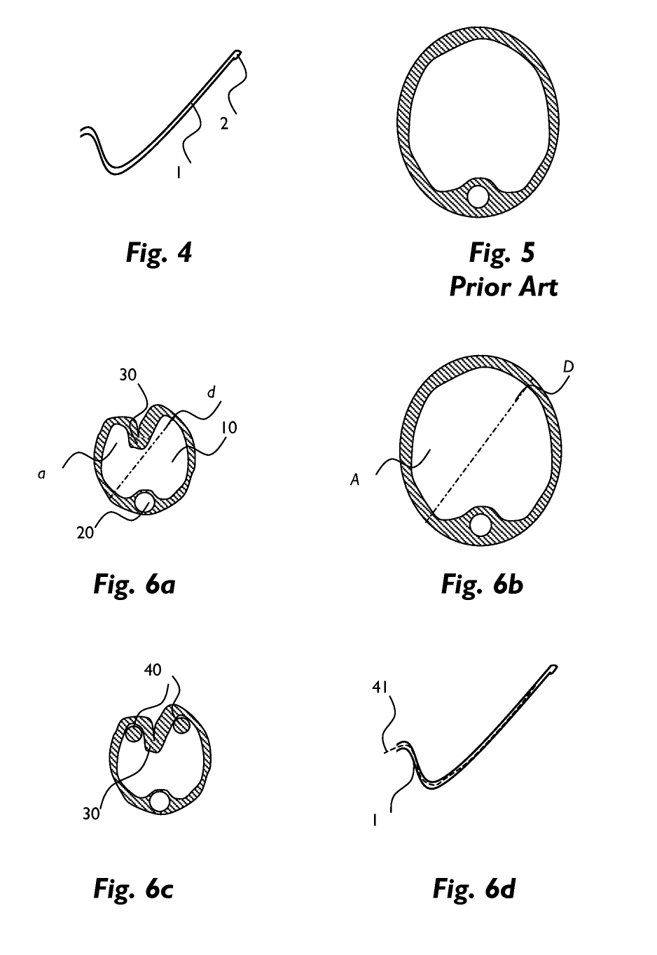

Reference is now made to FIG. 5, illustrating a cross section of a commercially available catheter. This view, provided here in a not-in-scale manner, schematically presents one portion of the aforesaid elongated shaft. The cross section shows a relatively larger open bore, suitable for fluid delivery, and a smaller bore (see at the bottom of the cross section), especially adapted for inflating and deflating a balloon located at the tip of the catheter. The structure of the envelope (walls) of the catheter is substantially smooth, uni-layered, homogeneous and thin, such that the catheter is flexible and characterized by a large diameter.

Reference is now made to FIG. 6a to FIG. 6d, schematically illustrating, still in a non-in-scale manner, transverse cross sections (FIG. 6a to FIG. 6c) and a sagittal cross section (FIG. 6d) of the novel catheter as defined in the present invention. FIG. 6a illustrates the hereby patented catheter in its collapsible configuration. Its smaller diameter and relative rigidity makes it easy for the physician to introduce the catheter within a cavity of the body. FIG. 6a thus shows the two sections of the catheter's lumen, namely the relatively large open bore 10, suitable for a non-complete fluid delivery, and a smaller bore 20 adapted for inflating and deflating a balloon located at the tip of the catheter. The structure of the envelope (walls) of the catheter in its collapsible configuration is textured (see vaginated sector 30), comprises various layers, is heterogeneous and respectfully thick, such that the catheter is somewhat rigid and characterized by a small diameter.

It is well in scope of the invention wherein the catheter comprises more than one vaginated sector. Hence for example, and in a non-limiting manner, according to one embodiment of the invention, one portion of the shaft comprises two vaginated sectors, the first in a 3 o'clock position and the second in a 9 o'clock position. Similarly, according to still another embodiment of the invention, one portion of the shaft may comprise three vaginated sectors, the first in a 9 o'clock position, the second in a 12 o'clock position and the third in a 3 o'clock position. According to another embodiment of the invention, one portion of the shaft may comprise one vaginated sector, e.g., in 12 o'clock; and in another portion of the shaft may comprising two vaginated sectors, e.g., in a 9 o'clock position and the third in a 3 o'clock position. The size of the vaginated sectors, i.e., the length of the vaginated envelope and hence, the area of the encapsulated envelope, may very. Hence, in at least one first portion of the shaft the amount of vaginated sectors is N and the area of the encapsulated envelope is M², whereas in at least one second portion of the shaft the amount of vaginated sectors is N′ and the area of the encapsulated envelope is M′²; N is equal or different from N′, and M is equal or different from M′. It is thus in the scope of the invention wherein the catheter has two or more collapsed configurations and/or two or more expand configurations.

Reference is now made to FIG. 6c schematically illustrating a catheter with a vaginated sector 30, effectively reversibly clutched by insert 40. Here, the insert comprises two wires which mechanically grasp the folded vagination in the collapsed configuration. According to one embodiment of the intervention, by pulling the wires or otherwise manipulating (physically, mechanically, electrically etc) or maneuvering the insert, the wires disengage and the catheter spontaneously turns into its expanded configuration. Still referring to a mechanical wire-based embodiment of the insert, the two wires may be secured in its clutched configuration in a safety-pin lock mechanism provided in the very proximal portion of the catheter (See 41 in FIG. 6d); by a zip-lock mechanism, by untying temporary sawed sutures, or by any other suitable mechanical, chemical (ungluing, dissolving etc) or electrical mechanism (e.g., compositions comprising shape memory alloys, such as nickel titanium (Nitinol) and/or electroactive polymers, such as ferroelectric polymers, conductive polymers, ionic polymer-metal composites, electrorheological fluids, ionic polymer-metal composites etc). It is hence n the scope of the invention wherein the insert and its parts (see e.g., wires 40) are at least partially made of polymers, metal ware etc.

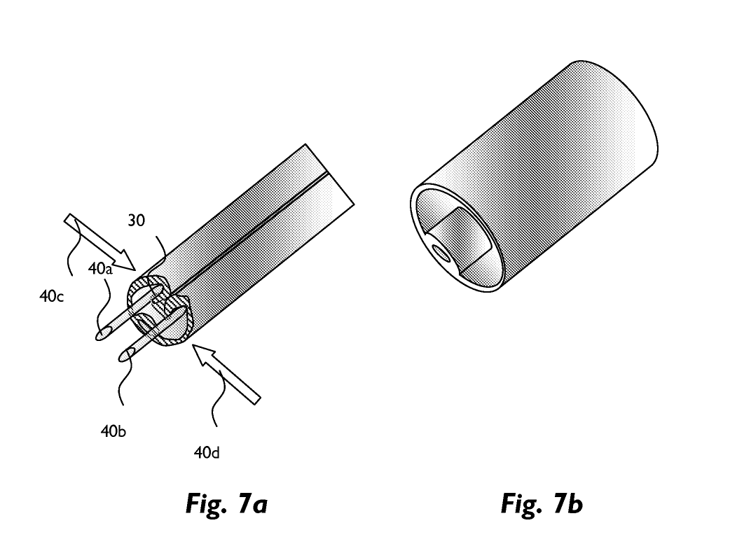

Reference is now made to FIG. 7a and FIG. 7b, schematically illustrating, still in a non-in-scale manner, the novel catheter as defined in the present invention, wherein FIG. 7a presents the collapsed configuration wherein two inserts, 40a and 40b effectively and continuously clasps the vagination 30 in strong opposite directions 40c and 40d, respectively. By pooling or opening the spring-like inserts, the vagination disappears (See FIG. 7b).

It is in the scope of the invention wherein the mechanical properties of the said portion of the shaft is selected from a group consisting of Young's modulus ranging e.g., from about 1,500 to about 15,000 lbf to in² and Shear modulus ranging e.g., about 0.01 to about 0.1 GPa.

While the invention is susceptible to various modifications and alternative forms, specific embodiments thereof have been shown by way of example in the drawings and will herein be described in detail. It should be understood, however, that it is not intended to limit the invention to the particular forms disclosed, but on the contrary, the intention is to cover all modifications, equivalents, and alternatives falling within the spirit and scope of the invention as defined by the appended claims.

What is claimed is:

1. A catheter comprising a continuous envelope forming an open-bore shaft; said envelope is characterized by a main longitudinal axis and a sagittal cross section; wherein at least one of the following is being held true:
a. at least a portion of said sagittal cross section is characterized by at least one asymmetric property; further wherein said asymmetric property extends along said main longitudinal axis according to predetermined pattern;
b. at least one cross section of said shaft and/or tip comprises at least one reversibly invaginated sector, such that said catheter is characterized by (i) at least one collapsible configuration of a relatively rigid shaft and/or tip and small cross section, and (ii) at least one expandable configuration of a flexible shaft and larger cross section;
c. at least a portion of said corneal cross section is characterized by at least one asymmetric property; further wherein said asymmetric property extends along said main longitudinal axis according to predetermined pattern;
d. at least a portion of said transverse cross section is characterized by at least one asymmetric property; further wherein said asymmetric property extends along said main longitudinal axis according to predetermined pattern; and
e. said envelope is characterized by a main longitudinal axis and a (i) sagittal cross section; (ii) corneal cross section; (iii) transverse cross section or any combination thereof, wherein at least a portion of either (i) said sagittal cross section; (ii) corneal cross section; (iii) transverse cross section or any combination thereof, is characterized by at least one asymmetric property; further wherein said asymmetric property extends along said main longitudinal axis according to predetermined pattern.

2. The catheter according to either one of claim 1, wherein at least one of the following is being held true:
a. said predetermined pattern is selected from a group consisting of parallel manner or a non-parallel manner;
b. said predetermined pattern is a coiled or helix like pattern in either clock wise or counter clock wise manner;
c. said catheter is insertable into a non-uniform body cavity;
d. said portion of said cross section comprises at least one bulge protruding either inwardly or outwardly of said envelope or any combination thereof; said
e. said portion of said envelope is made of material having predetermined Property Y1 while said envelope is made of material having predetermined Property Y2; where Y1 is substantially different than Y2.

3. The catheter according to claim 2, wherein at least one of the following is being held true:
a. said protruding bulge protrudes to a range of about 0.01 millimeters to about 2.5 millimeters;
b. the thickness of said at least one bulge is substantially different than the thickness of said envelope;
c. said bulge is located on the perimeter of said envelope;
d. said Property is selected from a group consisting of Young's modulus, rigidity, flexibility, shear modulus G, bulk modulus K, Poisson's ratio v, spring modulus, bending stiffness El, thickness, texture, composition, electrical conductivity, density, composition, elasticity;
e. said material is selected from a group consisting of Latex™, silicon containing material, shape memory materials, electro active materials or any combination thereof;
f. said envelope is made of at least one material selected from a group consisting of Latex™, silicon containing material, shape memory materials, electro active materials or any combination thereof;
g. said portion of said cross section comprises at least one balloon-style bulge, such that said asymmetric property is provided by wither inflating of deflating said balloon;
h. said asymmetric property is either automatically controlled or manually controlled by the user;
i. said portion of said cross section comprises either at least one balloon-style bulge; or at least one bulge or any combination thereof;
j. said asymmetric property is provided by means of predefined microstructure texture;
k. said at least one property is selected from a group consisting of size, texture, material from which said envelope is made of, mechanical properties, mechanical properties selected from a group consisting of rigidity, flexibility, Young's modulus E, shear modulus G, bulk modulus K, Poisson's ratio v, spring modulus, bending stiffness El, thickness, texture, composition, electrical conductivity, density, composition, elasticity or any combination thereof;
l. a linear reciprocation and/or rotatable maneuver of the proximal end of said shaft translates into movement of said catheter in said body cavity.

4. A method of treating a patient, said method comprising step of:
a. obtaining a catheter having a continuous envelope which forms an elongated open-bore shaft; said envelope is characterized by a main longitudinal axis and a sagittal cross section;
b. asymmetrically modifying at least one property in at least a portion of said sagittal cross section;
c. exceeding said section along said shaft according to predetermined pattern; and,
d. inserting said catheter into a body cavity; and,
e. treating said patient.

5. A method of treating a patient, said method comprising step of:
a. obtaining a catheter having a continuous envelope which forms an elongated open-bore shaft; said envelope is characterized by a main longitudinal axis and a corneal cross section;
b. asymmetrically modifying at least one property in at least a portion of said corneal cross section;
c. exceeding said section along said shaft according to predetermined pattern; and,
d. inserting said catheter into a body cavity; and,
e. treating said patient.

6. A method of treating a patient, said method comprising step of:
a. obtaining a catheter having a continuous envelope which forms an elongated open-bore shaft; said envelope is characterized by a main longitudinal axis and a transverse cross section;
b. asymmetrically modifying at least one property in at least a portion of said transverse cross section;
c. exceeding said section along said shaft according to predetermined pattern; and,
d. inserting said catheter into a body cavity; and,
e. treating said patient.

7. A method of treating a patient, said method comprising step of:
a. obtaining a catheter having a continuous envelope which forms an elongated open-bore shaft; said envelope is characterized by a main longitudinal axis and a (i) transverse cross section; (ii) sagittal cross section; (iii) sagittal cross section;
b. asymmetrically modifying at least one property in at least a portion of either (i) (i) transverse cross section; (ii) said sagittal cross section; (iii) said sagittal cross section; or any combination thereof;
c. exceeding said section along said shaft according to predetermined pattern; and,
d. inserting said catheter into a body cavity; and,
e. treating said patient.

8. The catheter of claim 1, wherein said at least one sector reversibly is held invaginated in said collapsible configuration by means of at least one insert.

9. A catheter having a continuous envelope forming an elongated open-bore shaft and/or tip insertable within a body cavity; the cross section of said shaft is reciprocally maneuverable from at least one collapsible configuration of diameter d to at least one expandable configuration of diameter D, wherein D>d.

10. A catheter having a continuous envelope forming an elongated open-bore shaft and/or tip insertable within a body cavity; the cross section of said shaft is reciprocally maneuverable from at least one collapsible configuration of cross section area a to at least one expandable configuration of cross section area A, wherein A>a.

11. The catheter of either claim 9 or 10, wherein said at least one portion of the shaft and/or tip characterized by a collapsible cross section is reversibly held in its collapsed/expended configuration by means of at least one insert.

12. A method of producing a catheter as defined in claim 1, comprising steps of providing a flexible polymer, especially silicon, rubber, latex or the like, through a pre-shaped extruder, such that at least one invaginated sector is obtained.

13. A method of treating a patient, said method comprising step of:
a. obtaining a catheter having a continuous envelope which forms an elongated open-bore shaft;
b. providing at least one cross section of said shaft with one or more reversibly invaginated sectors;
c. inserting said catheter, in its at least one collapsible configuration, within a body cavity whilst maintaining said shaft and/or tip with a relative rigidity and small cross section; and then, still within said cavity;
d. expanding the cross section of at least one portion of said catheter to its expanded configuration whilst maintaining said shaft and/or tip flexible and with larger cross section.

14. The method of claim 13, additionally comprising steps selected from (i) maintaining said shaft and/or tip in its collapsible configuration by providing an insert to effectively clutch the invaginated envelope within said sector, and (ii) expanding said shaft to its expanded configuration by manipulating said insert to stop clutching said invaginated envelope, such that a flexible shaft with large cross section is obtained.

15. A method of treating a patient, said method comprising step of:
a. obtaining a catheter having a continuous envelope which forms an elongated open-bore shaft;
b. providing at least one portion of said shaft with a reciprocal maneuverability to change from at least one collapsed configuration to at least one expand configuration, by providing said at least one portion with at least one collapsible configuration, characterized by a cross section area a, and at least one expandable configuration characterized by cross section area A; A>a;
c. inserting said catheter, in its at least one collapsible configuration, within a body cavity whilst maintaining said shaft and/or tip; and
d. expanding the cross section of at least one portion of said catheter to its expanded configuration whilst maintaining said shaft and/or tip flexible and with larger cross section.

16. The method of claim 15, additionally comprising steps selected from (i) maintaining said shaft and/or tip in its collapsible configuration by providing an insert to effectively clutch said portion of the shaft, and (ii) expanding said shaft to its expanded configuration by manipulating said insert to stop clutching, such that a flexible shaft with large cross section is obtained.

17. A method of treating a patient, said method comprising step of:
a. obtaining a catheter having a continuous envelope which forms an elongated open-bore shaft;
b. providing at least one portion of said shaft and/or tip with a reciprocal maneuverability to change from at least one collapsed configuration to at least one expand configuration, by providing said at least one portion with at least one collapsible configuration, characterized by diameter d, and at least one expandable configuration characterized by diameter D; D>d;
c. inserting said catheter, in its at least one collapsible configuration, within a body cavity whilst maintaining said shaft with a relative rigidity and small cross section; and then, still within said cavity;
d. expanding the cross section of at least one portion of said catheter to its expanded configuration whilst maintaining said shaft and/or tip flexible and with larger cross section.

18. The method of claim 17, additionally comprising steps selected from (i) maintaining said shaft and/or tip in its collapsible configuration by providing an insert to effectively clutch said portion of the shaft, and (ii) expanding said shaft to its expanded configuration by manipulating said insert to stop clutching, such that a flexible shaft and/or tip with large cross section is obtained.
